(19) United States
(12) Patent Application Publication
(10) Pub. No.: US 2026/0108704 A1
(43) Pub. Date: Apr. 23, 2026

(54) DRUG INJECTION DEVICE FOR HEART FAILURE

(71) Applicants: NANJING DRUM TOWER HOSPITAL, Jiangsu (CN); NANJING TECH UNIVERSITY, Jiangsu (CN)

(72) Inventors: Can XU, Jiangsu (CN); Dongjin WANG, Jiangsu (CN)

(21) Appl. No.: 19/155,395

(22) PCT Filed: Oct. 21, 2024
(86) PCT No.: PCT/CN2024/125983

§ 371 Date: Aug. 11, 2025

(30) Foreign Application Priority Data
Mar. 8, 2024 (CN) 202410264760.7

(51) Int. Cl. A61M 25/00; A61M 5/31; A61M 5/315

(52) U.S. Cl. CPC A61M 25/0084; A61M 5/315; A61M 25/0043; A61M 25/0075; A61M 2005/3128; A61M 2025/0076; A61M 2025/0085; A61M 2210/125

(57) ABSTRACT

The present invention discloses a drug injection device for heart failure, and key technical solutions thereof are as follows: The device includes a delivery catheter and an abutting component disposed at one end of the delivery catheter. Multiple guide holes are formed in the abutting component. A reservoir tube is slidably connected in the delivery catheter. Multiple injection needles in communication with the reservoir tube are disposed at one end of the reservoir tube close to the abutting component. The multiple injection needles are slidably inserted in the multiple guide holes respectively in one-to-one correspondence. A sealing plate is rotatably connected in an inner wall of one end of the reservoir tube close to the injection needle. The present invention has the following technical effects: By providing the reservoir tube, the present invention enables adjustment of the movable tube's extension length from the injection tube during operation. This controls the distance between the injection piston and the sealing plate, where a space volume between the injection piston and the sealing plate is a total dosage of the single-dose medication. In this way, accurate dose control is achieved for each injection.

### TECHNICAL FIELD

The present invention relates to the technical field of medical instruments, and in particular, to a drug injection device for heart failure.

### BACKGROUND

Heart failure refers to a disorder caused by impaired systolic or diastolic function of the heart. This results in the inability to sufficiently drain venous return blood from the body. Consequently, blood in the venous system is stagnated, and blood supply in the arterial system is insufficient, ultimately leading to circulatory system dysfunction.

Existing heart failure treatment options include medication, assistive devices, and heart transplantation. However, each approach faces significant challenges. For example, the medication generally causes recurrent symptoms in many patients, biventricular pacing therapy is not suitable for all patients, and even some patients fail to meet the physiological requirements for treatment and develop adverse reactions. In addition, when the treatment is performed by heart transplantation, the supply of donor hearts is extremely limited.

To address the problems, such as significant trauma of thoracotomy, low injection efficiency and suboptimal therapeutic outcomes in an existing clinical operation process, Chinese Patent CN113577504A discloses a heart failure treatment system, “including a control mechanism, a delivery catheter connected to the control mechanism, and an injection module disposed in the delivery catheter, where a distal end part of the injection module has a preset form; and a guiding and positioning apparatus, the guiding and positioning apparatus being at least partially disposed in the delivery catheter”.

In clinical application, because the injection module has a fixed length and lacks an appropriate dosing component, quantitative injection may only be determined according to the physician's operational experience, potentially leading to dosing variations. In addition, after the injection module performs an injection, a residual volume of medication remains in the injection module, and the remaining medication affects the next injection dose of the medications. Consequently, the above injection module may only perform a single injection operation. Therefore, it is very necessary to provide a drug injection device for heart failure to solve the above problems.

### SUMMARY

An objective of the present invention is to provide a drug injection device for heart failure with the following advantages: By providing a reservoir tube, the present invention enables adjustment of a movable tube's extension length from an injection tube during operation. This controls the distance between an injection piston and a sealing plate, where a space volume between the injection piston and the sealing plate is a total dosage of the single-dose medication. In this way, accurate dose control is achieved for each injection.

The above technical objective of the present invention is achieved through the following technical solutions: Provided is a drug injection device for heart failure, including a delivery catheter and an abutting component disposed at one end of the delivery catheter, where multiple guide holes are formed in the abutting component, a reservoir tube is slidably connected in the delivery catheter, multiple injection needles in communication with the reservoir tube are disposed at one end of the reservoir tube close to the abutting component, the multiple injection needles are slidably inserted in the multiple guide holes respectively in one-to-one correspondence, a sealing plate is rotatably connected in an inner wall of one end of the reservoir tube close to the injection needle, the sealing plate is perforated with multiple connecting holes in one-to-one correspondence to the multiple injection needles, the reservoir tube is provided with an opening at one end away from the sealing plate, a tube plug is rotatably connected to the reservoir tube at the opening, a limiting ring connected to the tube plug is fixedly connected to an inner wall of the delivery catheter close to the tube plug, an injection assembly is disposed on the tube plug, and a connecting assembly is disposed between the injection assembly and the sealing plate; and
the injection assembly includes an injection tube slidably extending through the tube plug, a movable tube is in threaded connection to an outer side of one end of the injection tube towards the reservoir tube, a positioning ring is connected to an outer side of the movable tube close to the reservoir tube, an end of the movable tube is fixedly connected to an injection piston, a liquid inlet hole in communication with the injection tube is formed in a center of the injection piston, multiple clamping blocks are fixedly connected around an inner wall of the reservoir tube close to the sealing plate, multiple clamping slots matched with the clamping blocks are formed around a side of the injection piston towards the sealing plate, an administration tube is detachably connected to one end of the injection tube away from the injection piston, a first check valve is mounted on the administration tube, a balance tube sequentially extending through the tube plug and the injection piston is disposed above the injection tube, one end of the balance tube away from the injection piston communicates with a connecting tube, an outer side of the connecting tube is fixedly connected to a second check valve, and an opening of the connecting tube is adapted to a syringe.

The present invention is further configured such that: the connecting assembly includes a connecting strip fixedly connected to a center of a side of the sealing plate away from the injection needle, a length of the connecting strip is greater than a length of the reservoir tube, one end of the connecting strip away from the sealing plate extends through the liquid inlet hole to be slidably connected in the movable tube, limiting grooves are formed on top and bottom sides in the movable tube and a liquid inlet tube, and two ends of the limiting groove respectively communicate with one end away from the injection piston and one end away from the movable tube.

The present invention is further configured such that: a sealing ring is fixedly connected to an inner side of the reservoir tube, the sealing ring rotatably sleeves an outer side of the sealing plate, and a side of the sealing ring opposite to the injection piston is flush with the sealing plate.

The present invention is further configured such that: ends of the multiple guide holes away from the reservoir tube diffuse to a direction away from a center of a circle of the abutting component.

The present invention is further configured such that: multiple through holes are formed at equal intervals along a length direction of the connecting strip, and a thickness of the connecting strip is less than a diameter of the liquid inlet hole.

The present invention is further configured such that: multiple support blocks are rotatably connected to an inner wall of the delivery catheter at equal intervals on a side of the limiting ring away from the reservoir tube, and the support blocks are provided with inserting holes for the injection tube and the balance tube to slide through.

The present invention is further configured such that: an outer side of one end of the administration tube close to the injection tube is sleeved with an extruding plate, and a diameter of the extruding plate is greater than a diameter of the injection tube.

The present invention is further configured such that: a diameter of the abutting component is the same as an outer diameter of the delivery catheter, and an edge of one end of the abutting component away from the delivery catheter is provided with a rounded corner.

The present invention is further configured such that: a friction force between the injection piston and an inner wall of the reservoir tube is less than a friction force between an outer wall of the reservoir tube and an inner wall of the delivery catheter.

The present invention has the following technical effects and advantages:
1. By providing the reservoir tube, the present invention enables adjustment of the movable tube's extension length from the injection tube during operation. This controls the distance between the injection piston and the sealing plate, where a space volume between the injection piston and the sealing plate is a total dosage of the single-dose medication. In this way, accurate dose control is achieved for each injection.
2. By providing the balance tube and the reservoir tube, the present invention enables temporary storage of each injection dose in the reservoir tube. This allows for precise control over medication dosage when multiple injections are required, so as to prepare for subsequent injections without removing the injection tube, thereby enabling repeated injections. In addition, when another medication needs to be injected for combination therapy, gas can be injected into the balance tube to vent residual medication from the previous injection remaining in the injection tube via the administration tube. This prevents over-injection of the previous medication, and improves the injection efficiency.

### BRIEF DESCRIPTION OF THE DRAWINGS

FIG. 1 is a schematic view of an overall structure of the present invention;

FIG. 2 is a cross-sectional view of an overall structure of the present invention;

FIG. 3 is an enlarged view of a part A in FIG. 2 according to the present invention;

FIG. 4 is a three-dimensional cross-sectional view of a reservoir tube according to the present invention;

FIG. 5 is an enlarged view of a part B in FIG. 4 according to the present invention;

FIG. 6 is a three-dimensional schematic view of an injection assembly, a sealing plate, a connecting strip, an extruding plate and other structures according to the present invention; and

FIG. 7 is a three-dimensional schematic view of an injection piston and a movable tube according to the present invention.

### REFERENCE NUMERALS

1, delivery catheter; 2, abutting component; 3, guide hole; 4, reservoir tube; 5, injection needle; 6, sealing plate; 7, connecting hole; 8, tube plug; 9, limiting ring; 10, injection tube; 11, movable tube; 12, positioning ring; 13, injection piston; 14, liquid inlet hole; 15, clamping block; 16, clamping slot; 17, administration tube; 18, first check valve; 19, balance tube; 20, connecting tube; 21, second check valve; 22, connecting strip; 23, limiting groove; 24, sealing ring; 25, through hole; 26, support block; and 27, extruding plate.

### DETAILED DESCRIPTION

The present invention is further described in detail below with reference to the accompanying drawings.

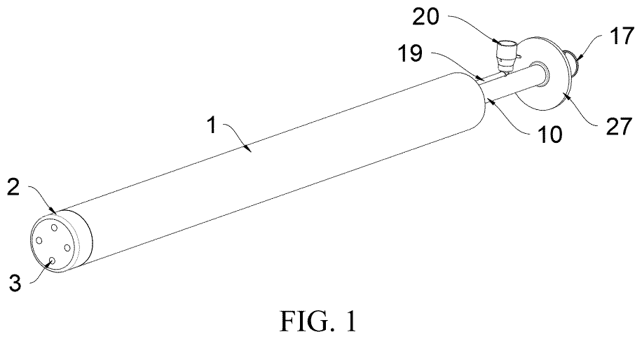

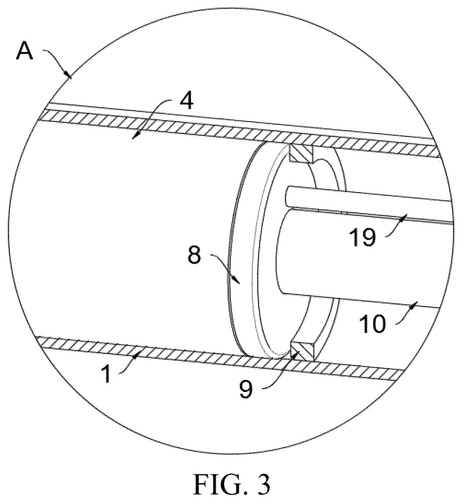

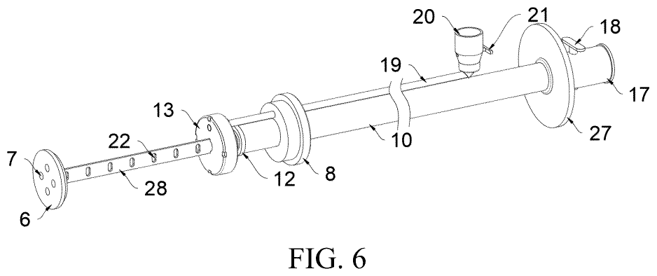

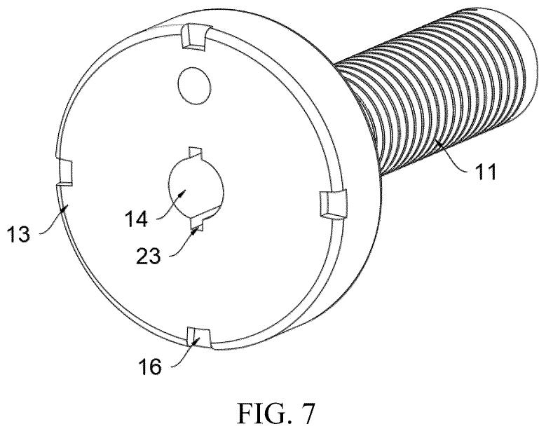

Embodiments: Referring to FIG. 1 to FIG. 7, provided is a drug injection device for heart failure, including a delivery catheter 1. An abutting component 2 is connected to one end of the delivery catheter 1, and the abutting component 2 is perforated with multiple guide holes 3. A diameter of the abutting component 2 is the same as an outer diameter of the delivery catheter 1, and an edge of one end of the abutting component 2 away from the delivery catheter 1 is provided with a rounded corner. A reservoir tube 4 is slidably mounted in the delivery catheter 1. Multiple injection needles 5 are connected to one end of the reservoir tube 4 close to the abutting component 2, the injection needles 5 communicate with the inside of the reservoir tube 4, and the multiple injection needles 5 are slidably inserted into the multiple guide holes 3 in one-to-one correspondence. A sealing plate 6 is rotatably mounted on an inner wall of one end of the reservoir tube 4 close to the injection needle 5. The sealing plate 6 is perforated with multiple connecting holes 7, and the positions of the multiple connecting holes 7 are in one-to-one correspondence to the positions of the multiple injection needles 5. The other end of the reservoir tube 4 is designed with an opening, and a tube plug 8 is rotatably connected to the open end of the reservoir tube 4. An injection assembly is slidably inserted into the tube plug 8. A connecting assembly is disposed between the injection assembly and the sealing plate 6. A limiting ring 9 is disposed on a side of the tube plug 8 away from the injection needle 5. The limiting ring 9 is fixedly connected to an inner wall of the delivery catheter 1.

The injection assembly includes an injection tube 10. The injection tube 10 slidably extends and is inserted into the tube plug 8. A movable tube 11 is in threaded insertion into one end of the injection tube 10 located in the reservoir tube 4. A positioning ring 12 is fixedly connected to an edge of one end of the movable tube 11 located outside the injection tube 10. An injection piston 13 is connected to one end of the movable tube 11 away from the injection tube 10, and a friction force between the injection piston 13 and an inner wall of the reservoir tube 4 is less than a friction force between an outer wall of the reservoir tube 4 and an inner wall of the delivery catheter 1. A center of the injection piston 13 is perforated with a liquid inlet hole 14, and the liquid inlet hole 14 communicates with the injection tube 10. Multiple clamping blocks 15 are fixedly connected to the inner wall of the reservoir tube 4, the clamping block 15 is close to a side of the sealing plate 6 close to the injection piston 13, and the multiple clamping blocks 15 are annularly distributed on the inner wall of the reservoir tube 4 evenly. A clamping slot 16 is formed in an edge position of a side of the injection piston 13 opposite to the multiple clamping blocks 15 respectively, a length of the clamping slot 16 is greater than a thickness of the clamping block 15, and a depth of the clamping slot 16 is greater than a height of the clamping block 15. An administration tube 17 is detachably connected to one end of the injection tube 10 away from the injection piston 13, and the administration tube 17 is provided with a first check valve 18. A balance tube 19 is disposed at the top of the injection tube 10. The balance tube 19 sequentially extends through and is inserted into the tube plug 8 and the injection piston 13. The balance tube 19 is detachably connected to the injection piston 13. A connecting tube 20 is detachably connected to one end of the balance tube 19 away from the injection piston 13, a second check valve 21 is disposed on the connecting tube 20, and a diameter of the connecting tube 20 is matched with a diameter of a port of an existing syringe. The connecting assembly includes a connecting strip 22, the connecting strip 22 is vertically and fixedly connected to a center of a side of the sealing plate 6 away from the injection needle 5, and a length of the connecting strip 22 is greater than a length of the reservoir tube 4. One end of the connecting strip 22 away from the sealing plate 6 is slidably inserted into the movable tube 11 through the liquid inlet hole 14 of the injection piston 13. Limiting grooves 23 in communication with each other are formed on the top and bottom inner walls of the movable tube 11 and the liquid inlet hole 14, and two ends of the limiting groove 23 respectively communicate with one end away from the injection piston 13 and one end away from the movable tube 11.

Before injection is performed, the injection assembly is adjusted first. Specific adjustment steps are as follows:
Step 1: The injection tube 10 is pushed, so that the injection tube drives the injection piston 13 to be attached to the sealing plate 6.
Step 2: The injection tube 10 and the balance tube 19 are twisted, so that the injection piston 13 is driven by the injection tube 10 and the balance tube 19 to rotate. When the multiple clamping slots 16 on the injection piston 13 respectively correspond to the multiple clamping blocks 15 on the inner wall of the reservoir tube 4, the injection piston 13 is pushed again through the injection tube 10 and the balance tube 19, so that the clamping slots 16 on the injection piston 13 and the corresponding clamping blocks 15 are stuck together.
Step 3: The injection tube 10 is twisted. In this case, because the injection piston 13 is limited by the clamping block 15 on the inner wall of the reservoir tube 4, as the injection tube 10 rotates, the movable tube 11 can gradually extend from the injection tube 10, and the injection tube 10 can move away from the injection piston 13. As the distance of the movable tube 11 extending from the injection tube 10 increases, when the positioning ring 12 at the end of the injection tube 10 is pulled to be in contact with the tube plug 8, a space volume between the injection piston 13 and the sealing plate 6 is a total dosage of the single-dose medication. In this way, accurate dose control is achieved for each injection.
Step 4: After the adjustment of the movable tube 11, the injection tube 10 is pulled to disengage the injection piston 13 from the clamping block 15, and then, the injection tube 10 and the balance tube 19 continue to be twisted. Because the injection piston 13 is not limited by the clamping block 15 in this case, as the injection tube 10 and the balance tube 19 are twisted, the injection piston 13 drives the connecting strip 22 and the sealing plate 6 to rotate together through the limiting groove 23, so that the multiple connecting holes 7 on the sealing plate 6 keep in communication with the multiple injection needles 5. At this point, the adjustment of the injection assembly is completed.

During an injection operation, first, two bent sheathing canals (where the sheathing canals are commonly used guiding and positioning devices in conventional interventional therapies) pass through the femoral artery, pass through the aortic arch accordingly, pass through the aortic valve position after being bent through the first layer of bent sheathing canals, then reach the left ventricle, and finally pass through the second sheathing canal to vertically press the distal end thereof against the myocardial tissue. Then, the delivery catheter 1 is inserted into the second sheathing canal to enable the abutting component 2 at the end of the delivery catheter 1 to be vertically abutted to the myocardial tissue under the guiding action of the second sheathing canal.

Subsequently, injection judgment is performed. Steps of the injection judgment are as follows:
Step 1: The second check valve 21 is opened on the premise that the first check valve 18 is closed, and then, the injection tube 10 and the balance tube 19 are pushed, so that the injection tube and the balance tube drive the injection piston 13 to move close to the sealing plate 6. In this process, as the injection piston 13 moves in the reservoir tube 4, the air in the reservoir tube 4 can be vented through the balance tube 19. When the injection piston 13 is in contact with the sealing plate 6, the injection tube 10 and the balance tube 19 continue to be pushed, so that the reservoir tube 4 is pushed by the injection tube 10 and the balance tube 19 to drive the injection needle 5 to move close to the myocardial tissue.
Step 2: With continued movement of the reservoir tube 4, the injection needle 5 gradually extends from the guide hole 3. When the reservoir tube 4 is in contact with the abutting component 2, pushing of the injection tube 10 and the balance tube 19 is stopped.
Step 3: Subsequently, the existing syringe is connected to the connecting tube 20, and an air extraction action is performed. If the syringe can be reset, it is proved that the injection needle 5 is inserted into the myocardial tissue, and in this case, the subsequent injection operation can be performed. If the syringe cannot be reset, it indicates that the injection needle 5 is not inserted into the myocardial tissue, and the position of the abutting component 2 needs to be adjusted again, so as to facilitate injection again.

When it is determined that injection can be performed, the injection tube 10 is pulled first, so that the injection tube drives the positioning ring 12 to be in contact with the tube plug 8, and in this case, the injection needle 5 is still inserted into the myocardial tissue. Subsequently, the injection tube 10 is twisted first, so that the injection tube drives the sealing plate 6 to rotate through the connecting strip 22, thereby sealing the injection needle 5. Subsequently, the first check valve 18 is opened, and the medication is injected into the injection tube 10 through the administration tube 17. In this case, because the injection needle 5 is sealed, the medication entering the injection tube 10 can enter the reservoir tube 4 through the liquid inlet hole 14. As the medication gradually increases in the reservoir tube 4, the air in the reservoir tube 4 can be vented through the balance tube 19. When the reservoir tube 4 is filled with the medication, a particular dose of the medication continues to be injected into the reservoir tube 4, and the redundant medication can enter the balance tube 19. The purpose of this operation is that the air in the reservoir tube 4 is completely vented, so as to prevent the air from being injected into the myocardial tissue. After filling of the medication in the reservoir tube 4, the first check valve 18 and the second check valve 21 are closed first, and then, the injection tube 10 is twisted to drive the sealing plate 6 to rotate through the connecting tube 20, so that the connecting hole 7 in the sealing plate 6 communicates with the injection needle 5, and then, the injection tube 10 is pushed. In this case, the injection piston 13 injects the whole medication in the reservoir tube 4 into the myocardial tissue under the push of the injection tube 10, thereby implementing a quantitative injection operation.

When the second injection needs to be performed, the medication can be added to the reservoir tube 4 according to the above medication adding manner. After the addition of the medication, the injection tube 10 can be pushed to perform the second injection, and the dose of the medication in each injection is the same. In addition, repeated injections can also be implemented in the above operation manner.

In addition, when another medication needs to be injected for cooperative treatment, to avoid that the previous medication remains in the injection tube 10, the first check valve 18 and the second check valve 21 can be opened at the same time while the sealing plate 6 is kept closed, and then, gas is injected into the connecting tube 20. With the injection, the gas can enter the injection tube 10 through the reservoir tube 4, so that the medication remaining from the previous injection is vented via the administration tube 17. This prevents over-injection of the previous medication, and improves the injection efficiency without injection again.

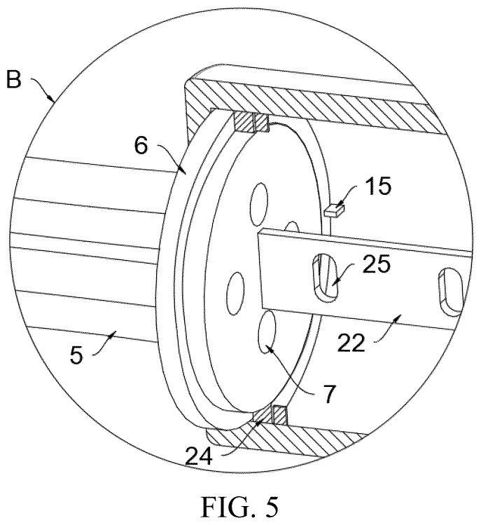

As shown in FIG. 5, an outer side of the sealing plate 6 is rotatably sleeved with a sealing ring 24, the sealing ring 24 is fixedly connected to the inner wall of the reservoir tube 4, and a side of the sealing ring 24 close to the injection piston 13 is flush with the sealing plate 6.

By providing the sealing ring 24, the sealing ring 24 can improve the sealing performance between a side surface of the sealing plate 6 and an inner wall of the reservoir tube 4, thereby avoiding the condition that when the medication is injected into the reservoir tube 4, the medication flows from the reservoir tube 4 in advance through a gap between the sealing plate 6 and the inner wall of the reservoir tube 4, and also ensuring the accuracy of detecting whether the injection needle 5 is inserted into the myocardial tissue through a syringe.

As shown in FIG. 1 to FIG. 4, a length of the guide hole 3 is less than a length of the injection needle 5, and ends of the multiple guide holes 3 away from the reservoir tube 4 diffuse to a direction away from a center of a circle of the abutting component 2.

When the multiple injection needles 5 are inserted into the myocardial tissue under the guiding action of the guide holes 3, the multiple injection needles 5 can perform an injection operation in a diffused form by taking the center of the abutting component 2 as a point, thereby increasing an injection area of the multiple injection needles 5 and improving the action effect and efficiency of the medication.

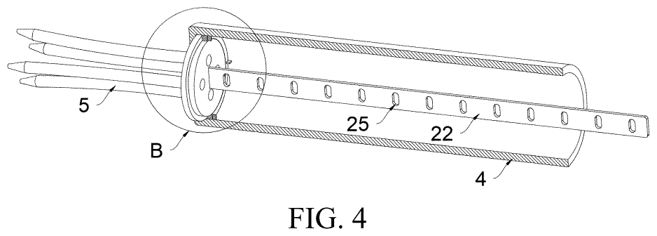

As shown in FIG. 4 to FIG. 7, a thickness of the connecting strip 22 is less than a diameter of the liquid inlet hole 14, and multiple through holes 25 are evenly perforated in the connecting strip 22 along the front-rear direction.

By forming the through holes 25 in the connecting strip 22, when the medication enters the movable tube 11 and the liquid inlet hole 14 through the injection tube 10, the through holes 25 in the connecting strip 22 can increase the speed at which the medication enters the reservoir tube 4 through the liquid inlet hole 14 on the premise that the connecting strip 22 is inserted into the liquid inlet hole 14, thereby ensuring the injection efficiency.

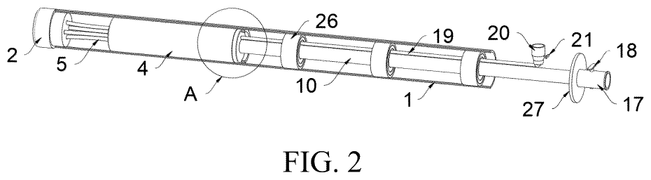

As shown in FIG. 2, multiple support blocks 26 are evenly distributed in the delivery catheter 1. The multiple support blocks 26 are located on a side of the limiting ring 9 away from the reservoir tube 4. The support blocks 26 are rotatably mounted on an inner wall of the delivery catheter 1. The support blocks 26 are perforated with inserting holes along directions of two sides of the support blocks, and the injection tube 10 and the balance tube 19 sequentially slidably extend and are inserted into the inserting holes of the multiple support blocks 26.

By providing the support blocks 26 in the delivery catheter 1, the multiple support blocks 26 can support and limit parts of the injection tube 10 and the balance tube 19 located in the delivery catheter 1. Therefore, when the injection piston 13 is rotated by twisting the injection tube 10 and the balance tube 19, the support blocks 26 can rotate with the injection tube 10 and the balance tube 19 by taking the center of the injection piston 13 as an axis, so that the injection tube 10 and the balance tube 19 can be always attached to each other in parallel under the restriction of the support blocks 26, thereby preventing the injection tube and the balance tube from winding. In addition, when the injection piston 13 is pushed through the injection tube 10, the evenly disposed support blocks 26 can support an outer wall of the injection tube 10, thereby preventing the injection tube 10 from being bent in a process of pushing the injection piston 13.

As shown in FIG. 1 and FIG. 2, one end of the administration tube 17 close to the injection tube 10 is fixedly sleeved with an extruding plate 27, and a diameter of the extruding plate 27 is greater than a diameter of the injection tube 10.

By providing the extruding plate 27, when the injection piston 13 is pushed through the injection tube 10 to perform an injection operation, the extruding plate 27 can increase a contact area between the hand of a medical worker and the administration tube 17, thereby improving the convenience of the injection operation.

The specific embodiments are only used for explaining the present invention but are not intended to limit the present invention. A person skilled in the art may make modifications to this embodiment without creative contribution according to requirements after reading this specification. However, as long as the embodiments fall within the scope of the claims of the present invention, the embodiments are protected by the patent law.

What is claimed is:

1. A drug injection device for heart failure, comprising a delivery catheter (1) and an abutting component (2) disposed at one end of the delivery catheter (1), wherein a plurality of guide holes (3) are formed in the abutting component (2), a reservoir tube (4) is slidably connected in the delivery catheter (1), a plurality of injection needles (5) in communication with the reservoir tube (4) are disposed at one end of the reservoir tube (4) close to the abutting component (2), the plurality of injection needles (5) are slidably inserted in the plurality of guide holes (3) respectively in one-to-one correspondence, a sealing plate (6) is rotatably connected in an inner wall of one end of the reservoir tube (4) close to the injection needle (5), the sealing plate (6) is perforated with a plurality of connecting holes (7) in one-to-one correspondence to the plurality of injection needles (5), the reservoir tube (4) is provided with an opening at one end away from the sealing plate (6), a tube plug (8) is rotatably connected to the reservoir tube (4) at the opening, a limiting ring (9) connected to the tube plug (8) is fixedly connected to an inner wall of the delivery catheter (1) close to the tube plug (8), an injection assembly is disposed on the tube plug (8), and a connecting assembly is disposed between the injection assembly and the sealing plate (6); and
the injection assembly comprises an injection tube (10) slidably extending through the tube plug (8), a movable tube (11) is in threaded connection to an outer side of one end of the injection tube (10) towards the reservoir tube (4), a positioning ring (12) is connected to an outer side of the movable tube (11) close to the reservoir tube (4), an end of the movable tube (11) is fixedly connected to an injection piston (13), a liquid inlet hole (14) in communication with the injection tube (10) is formed in a center of the injection piston (13), a plurality of clamping blocks (15) are fixedly connected around an inner wall of the reservoir tube (4) close to the sealing plate (6), a plurality of clamping slots (16) matched with the clamping blocks (15) are formed around a side of the injection piston (13) towards the sealing plate (6), an administration tube (17) is detachably connected to one end of the injection tube (10) away from the injection piston (13), a first check valve (18) is mounted on the administration tube (17), a balance tube (19) sequentially extending through the tube plug (8) and the injection piston (13) is disposed above the injection tube (10), one end of the balance tube (19) away from the injection piston (13) communicates with a connecting tube (20), an outer side of the connecting tube (20) is fixedly connected to a second check valve (21), and an opening of the connecting tube (20) is adapted to a syringe.

2. The drug injection device for heart failure according to claim 1, wherein the connecting assembly comprises a connecting strip (22) fixedly connected to a center of a side of the sealing plate (6) away from the injection needle (5), a length of the connecting strip (22) is greater than a length of the reservoir tube (4), one end of the connecting strip (22) away from the sealing plate (6) extends through the liquid inlet hole (14) to be slidably connected in the movable tube (11), limiting grooves (23) are formed on top and bottom sides in the movable tube (11) and a liquid inlet tube, and two ends of the limiting groove (23) respectively communicate with one end away from the injection piston (13) and one end away from the movable tube (11).

3. The drug injection device for heart failure according to claim 1, wherein a sealing ring (24) is fixedly connected to an inner side of the reservoir tube (4), the sealing ring (24) rotatably sleeves an outer side of the sealing plate (6), and a side of the sealing ring (24) opposite to the injection piston (13) is flush with the sealing plate (6).

4. The drug injection device for heart failure according to claim 1, wherein ends of the plurality of guide holes (3) away from the reservoir tube (4) diffuse to a direction away from a center of a circle of the abutting component (2).

5. The drug injection device for heart failure according to claim 2, wherein a plurality of through holes (25) are formed at equal intervals along a length direction of the connecting strip (22), and a thickness of the connecting strip (22) is less than a diameter of the liquid inlet hole (14).

6. The drug injection device for heart failure according to claim 1, wherein a plurality of support blocks (26) are rotatably connected to an inner wall of the delivery catheter (1) at equal intervals on a side of the limiting ring (9) away from the reservoir tube (4), and the support blocks (26) are provided with inserting holes for the injection tube (10) and the balance tube (19) to slide through.

7. The drug injection device for heart failure according to claim 1, wherein an outer side of one end of the administration tube (17) close to the injection tube (10) is sleeved with an extruding plate (27), and a diameter of the extruding plate (27) is greater than a diameter of the injection tube (10).

8. The drug injection device for heart failure according to claim 1, wherein a diameter of the abutting component (2) is the same as an outer diameter of the delivery catheter (1), and an edge of one end of the abutting component (2) away from the delivery catheter (1) is provided with a rounded corner.

9. The drug injection device for heart failure according to claim 1, wherein a friction force between the injection piston (13) and an inner wall of the reservoir tube (4) is less than a friction force between an outer wall of the reservoir tube (4) and an inner wall of the delivery catheter (1).
